(19) United States
(12) Patent Application Publication
(10) Pub. No.: US 2025/0009994 A1
(43) Pub. Date: Jan. 9, 2025

(54) AN INSUFFLATOR AND A METHOD FOR DETERMINING AN OPTIMUM MAXIMUM PRESSURE BEYOND WHICH A CAVITY IN THE BODY OF A HUMAN OR ANIMAL SUBJECT SHOULD NOT BE INSUFFLATED

(71) Applicant: PALLIARE LIMITED, Dangan, Galway (IE)

(72) Inventor: John O'DEA, Bearna (IE)

(73) Assignee: PALLIARE LIMITED, Dangan, Galway (IE)

(21) Appl. No.: 18/699,921

(22) PCT Filed: Jan. 12, 2023
(86) PCT No.: PCT/IE2023/000001

§ 371 Date: Apr. 10, 2024

(30) Foreign Application Priority Data
Jan. 12, 2022 (IE) S2022/0005

(51) Int. Cl. A61M 13/00

(52) U.S. Cl. CPC A61M 13/003; A61M 2202/0007; A61M 2202/02; A61M 2205/3327; A61M 2205/3334; A61M 2205/52

(57) ABSTRACT

An insufflator (1) operable in a set-up mode and a normal insufflating mode comprises a housing (10) for receiving pressurised insufflating gas from an external source (11) thereof. A microprocessor (13) controls a flow controller (16) in response to signals read from a pressure monitoring device (21) and a flow sensor (20) to maintain the peritoneal cavity (3) of a subject insufflated at a selectable working pressure. In the set-up mode, an optimum maximum pressure value is determined, beyond which the cavity (3) should ideally not be insufflated, since beyond the optimum maximum pressure there is minimal gain in working volume in the cavity (3), while the pressure in the cavity (3) increases significantly for each unit volume of insufflating gas delivered to the cavity (3). The optimum maximum pressure is determined as being the pressure in the cavity at the point of inflection (39) on a line (35) representing the pressure/volume relationship between the pressure in the cavity and the cumulative volume of insufflating gas delivered to the cavity (3) where a first linear relationship transitions to a second linear relationship. The point of inflection (39) is determined from a smoothed version (37) of the line (35) where the portions of the smoothed line (37) representing the first and second pressure/volume relationships intersect at (39).

The present invention relates to a method for insufflating a cavity in the body of a human or animal subject, and in particular, though not limited to the peritoneal cavity, and the invention also relates to a method for determining an optimum maximum pressure, beyond which a cavity in the body of a human or animal subject should ideally not be insufflated. The invention also relates to an insufflator, and in particular, to an insufflator which is configured for determining an optimum maximum pressure beyond which a cavity in the body of a human or animal subject should ideally not be insufflated.

Medical insufflators are used to create a pneumoperitoneum, namely, a working volume that allows a surgeon to operate laparoscopically in the peritoneal cavity, thereby providing a working volume therein for visualisation and manipulation of surgical tools and instruments. Current uses of insufflators require that surgeons select an insufflating pressure based on heuristic methods, for example, quoted from clinical literature. As insufflating gas is being delivered to the peritoneal cavity pressure increases within the cavity, and the cavity expands creating a working volume for the surgeon. However, the pressure required to achieve an adequate working volume varies, depending on the compliance of the peritoneal cavity. For example, in the case of a heavy subject with a lot of body fat, the peritoneal cavity will not expand to the same extent as the peritoneal cavity of a light subject with little or no body fat, therefore requiring a higher insufflating pressure than that normally quoted in the literature. Conversely, commonly quoted insufflating pressure values in the literature may be inappropriately high for a lighter, thinner subject with little or no body fat. In other words, adequate working volume in the peritoneal cavity could be achieved at a lower pressure in a lighter person than in a heavier person. Additionally, the position of a subject on an operating table may also influence the insufflating pressure required to gain an adequate working volume, and in turn visualisation, in the peritoneal cavity, for example, depending on the position of the subject on the operating table, the legs of a subject may squeeze the abdomen, thereby reducing the compliance of the peritoneal cavity.

Accordingly, based on heuristic methods, the peritoneal cavity, and other cavities in one subject may be insufflated to a pressure in excess of a pressure required for insufflating a similar cavity in another subject. In other cases, the peritoneal cavity or other cavity, may be insufflated in a subject to a pressure where the peritoneal cavity is becoming non-compliant to the extent that the delivery of further insufflating gas to the cavity would result in a significant increase in pressure in the cavity with little or no gain in the working volume of the cavity. It is desirable that during inflating of a cavity in a human or animal subject, the pressure in the cavity should be maintained at the lowest possible pressure consistent with providing an adequate working volume therein, in order to avoid any clinical risks, such as post operative pain, reduction in venous return, and other such complications which may arise when the cavity is insufflated to a pressure beyond a safe insufflating pressure.

There is therefore a need for an insufflator and a method which addresses this problem. In particular, there is a need for an insufflator which determines an optimum maximum pressure, beyond which a cavity, for example, a peritoneal cavity of a subject, should ideally not be inflated. There is also a need for a method for determining an optimum maximum pressure beyond which a cavity, for example, a peritoneal cavity in a human or animal subject should ideally not be inflated beyond.

The present invention is directed towards providing such an insufflator and a method.

According to the invention there is provided an insufflator adapted to be selectively operated in a normal insufflating mode and in a set-up mode, the insufflator being configured in the set-up mode for determining the value of an optimum maximum pressure for insufflating a cavity in the body of a human or animal subject, the insufflator comprising:
a delivery means for delivering insufflating gas to the cavity,
a pressure sensor for producing a signal indicative of the pressure in the cavity,
a flow sensor for monitoring flow of insufflating gas being delivered to the cavity and for producing a signal indicative of the cumulative volume of insufflating gas delivered to the cavity from the commencement of delivery of the insufflating gas thereto or a signal indicative of the rate at which the insufflating gas is being delivered to the cavity, and
a signal processor adapted to read the signal produced by the pressure sensor, and to read the signal produced by the flow sensor during insufflating of the cavity, and in the set-up mode:
to determine a pressure/volume relationship between the pressure in the cavity and insufflating gas delivered to the cavity as the insufflating gas is being delivered to the cavity from values of the signals read from the pressure sensor and the flow sensor, and
to determine the value of the optimum maximum pressure as the value of a transition pressure at which the pressure/volume relationship transitions from a first pressure/volume relationship to a second pressure/volume relationship, the second pressure/volume relationship being different to the first pressure/volume relationship.

In one embodiment of the invention the pressure/volume relationship determined by the signal processor comprises the value of the increase in pressure in the cavity per unit volume of insufflating gas delivered to the cavity.

In another embodiment of the invention the first pressure/volume relationship comprises either a substantially linear relationship or a non-linear relationship, and preferably, the first pressure/volume relationship comprises a substantially linear relationship during which the pressure in the cavity increases linearly with respect to the delivery of insufflating gas to the cavity, and in another embodiment of the invention the first pressure/volume relationship comprises a substantially linear relationship during which the increase in pressure in the cavity per unit volume of insufflating gas delivered to the cavity remains at a substantially constant value, and preferably, a substantially constant value greater than zero.

In another embodiment of the invention the second pressure/volume relationship comprises either a substantially linear relationship or a non-linear relationship.

In another embodiment of the invention the second pressure/volume relationship comprises a substantially linear relationship during which the pressure in the cavity increases linearly with respect to the delivery of insufflating gas to the cavity, and preferably, the second pressure/volume relationship comprises a substantially linear relationship during which the increase in pressure in the cavity per unit volume of insufflating gas delivered to the cavity remains at a substantially constant value, and in one embodiment of the invention the value of the increase in the pressure in the cavity per unit volume of insufflating gas delivered to the cavity in the second pressure/volume relationship, is greater than the value of the increase in the pressure in the cavity per unit volume of insufflating gas delivered to the cavity in the first pressure/volume relationship.

In another embodiment of the invention the first pressure/volume relationship transitions to the second pressure/volume relationship through an intermediate pressure/volume relationship, and preferably, the intermediate pressure/volume relationship comprises a non-linear relationship.

In one embodiment of the invention the signal processor is programmed to determine the transition pressure value as a pressure value lying in a range between a first pressure value and a second pressure value, and the signal processor is programmed to determine the first pressure value as the pressure at which the first pressure/volume relationship transitions to the intermediate pressure/volume relationship, and the signal processor is programmed to determine the second pressure value as the pressure at which the intermediate pressure/volume relationship transitions to the second pressure/volume relationship.

In one embodiment of the invention the signal processor is programmed to determine the transition pressure value as the average value of the first and the second pressure values.

In another embodiment of the invention the signal processor is programmed to determine the transition pressure value as a point of inflection on a line representative of a graph of the pressure/volume relationship during insufflating of the cavity, as the first pressure/volume relationship transitions to the second pressure/volume relationship.

In another embodiment of the invention in the graph of the pressure/volume relationship, volume is plotted on the abscissa, and pressure is plotted on the ordinate of the graph.

Preferably, the signal processor is programmed to determine the point of inflection by interpolating the point of intersection of a portion of the line representative of the first pressure/volume relationship and a portion of the line representative of the second pressure/volume relationship. Alternatively, the signal processor is programmed to determine the point of inflection on the line of the graph representative of the pressure/volume relationship during insufflating of the cavity by extrapolating the portion of the line representing the first pressure/volume relationship beyond the first pressure value, and extrapolating the line representing the second pressure/volume relationship beyond the second pressure value, and to determine the value of the transition pressure at the point of intersection of the extrapolated parts of the lines representing the first and second pressure/volume relationship.

In another embodiment of the invention the signal processor is programmed to read the values of the signals produced by the pressure sensor and the flow sensor either continuously or at predefined time intervals. Preferably, the signal processor is programmed to time-stamp, cross-reference and store in memory each pair of the values of the signals read from the pressure sensor and the flow sensor.

In one embodiment of the invention the signal processor is programmed to determine the value of the transition pressure from the stored, time-stamped and cross-referenced values of the pairs of values of the signals read from the pressure sensor and the flow sensor.

In another embodiment of the invention the signal processor is programmed to determine the value of the cumulative volume of insufflating gas delivered to the cavity and the corresponding value of the pressure in the cavity, each time the values of the signals are read from the flow sensor and the pressure sensor, and to time-stamp, cross-reference and store in memory each pair of the determined value of the cumulative volume of the insufflating gas delivered to the cavity and the corresponding value of the pressure in the cavity. Preferably, the signal processor is programmed to determine the value of the transition pressure from the stored, cross-referenced and time-stamped pairs of values of the cumulative volume of insufflating gas delivered to the cavity and the corresponding pressure in the cavity.

In one embodiment of the invention the signal processor is programmed to compute the value of the increase in the pressure in the cavity per unit volume of insufflating gas delivered to the cavity each time the values of the signals are read by the signal processor from the pressure sensor and the flow sensor. Preferably, the signal processor is programmed to apply a smoothing algorithm to the computation of each value of the increase in pressure in the cavity per unit volume of insufflating gas delivered to the cavity, and preferably, the smoothing algorithm comprises a moving average algorithm. Advantageously, the signal processor is programmed to time-stamp, cross-reference and store in memory each computed value of the increase in the pressure in the cavity per unit volume of insufflating gas delivered to the cavity and the corresponding value of the pressure in the cavity.

In one embodiment of the invention the signal processor is programmed to determine the value of the transition pressure from the computed values of the increase in pressure in the cavity per unit volume of insufflating gas delivered to the cavity and the corresponding values of the pressure in the cavity.

In one embodiment of the invention the signal processor is programmed to determine the first pressure value from the computed values of the increase in the pressure in the cavity per unit volume of the insufflating gas delivered to the cavity and the corresponding values of the pressure in the cavity.

In another embodiment of the invention the signal processor is programmed to determine the second pressure value from the computed values of the increase in the pressure in the cavity per unit volume of the insufflating gas delivered to the cavity and the corresponding values of the pressure in the cavity and the corresponding values of the pressure in the cavity.

In another embodiment of the invention the signal processor is programmed to store the value of the optimum maximum pressure in memory.

Preferably, the signal processor is programmed to produce a signal indicative of the value of the optimum maximum pressure. Preferably, the signal produced by the signal processor indicative of the value of the optimum maximum pressure is adapted for conversion to a human sensory perceptible signal.

Advantageously, the signal indicative of the value of the optimum maximum pressure produced by the signal processor is adapted for applying to a visual display screen for displaying the value of the optimum maximum pressure thereon.

In one embodiment of the invention the signal processor is programmed to terminate delivery of insufflating gas to the cavity in the set-up mode in response to the pressure in the cavity reaching a pressure beyond which the cavity can no longer be safely insufflated.

In another embodiment of the invention the signal processor is programmed to terminate delivery of insufflating gas to the cavity in the set-up mode in response to the cavity pressure reaching a maximum set-up safe pressure.

In another embodiment of the invention the maximum set-up safe pressure is either selectable or predefined.

In one embodiment of the invention the maximum set-up safe pressure lies in the range of 20 mmHg to 25 mmHg. Preferably, the maximum set-up safe pressure lies in the range of 10 mmHg to 15 mmHg. Advantageously, the maximum set-up safe pressure is approximately 15 mmHg.

In another embodiment of the invention the signal processor is programmed to terminate delivery of insufflating gas to the cavity in the set-up mode in response to the value of the transition pressure being determined.

In another embodiment of the invention the signal processor is programmed to terminate delivery of insufflating gas to the cavity in the set-up mode in response to the second pressure value being determined.

In one embodiment of the invention the signal processor is programmed to limit the supply of insufflating gas to the cavity in response to the pressure in the cavity reaching the optimum maximum pressure value when the insufflator is operating in the normal insufflating mode.

In another embodiment of the invention the signal processor is programmed to terminate the supply of insufflating gas to the cavity in response to the pressure in the cavity exceeding the optimum maximum pressure when the insufflator is operating in the normal insufflating mode.

In another embodiment of the invention the signal processor is programmed to terminate the supply of insufflating gas to the cavity in response to the pressure in the cavity reaching the optimum maximum pressure value when the insufflator is operating in the normal insufflating mode.

In one embodiment of the invention the signal processor is programmed to reinstate the supply of insufflating gas to the cavity in response to the pressure in the cavity falling below the optimum maximum pressure value when the insufflator is operating in the normal insufflating mode.

The invention also provides a method for determining an optimum maximum pressure value for insuflating a cavity in the body of a human or animal subject, the method comprising:
delivering insufflating gas to the cavity,
determining a pressure/volume relationship between the pressure in the cavity and insufflating gas delivered to the cavity as the insufflating gas is being delivered to the cavity,
determining the optimum maximum pressure value as the value of a transition pressure at which the pressure/volume relationship transitions from a first pressure/volume relationship to a second pressure/volume relationship, the second pressure/volume relationship being different to the first pressure/volume relationship.

In one embodiment of the invention the determined pressure/volume relationship comprises the value of the increase in pressure in the cavity per unit volume of insufflating gas delivered to the cavity.

In one embodiment of the invention the first pressure/volume relationship comprises either a substantially linear relationship or a non-linear relationship, and preferably, the first pressure/volume relationship comprises a substantially linear relationship during which the pressure in the cavity increases with respect to the delivery of insufflating gas to the cavity, and in another embodiment of the invention the first pressure/volume relationship comprises a substantially linear relationship during which the value of the increase in the pressure in the cavity per unit volume of insufflating gas delivered to the cavity remains at a substantially constant value, and preferably, at a substantially constant value greater than zero.

In another embodiment of the invention the second pressure/volume relationship comprises either a substantially linear relationship or a non-linear relationship.

In another embodiment of the invention the second pressure/volume relationship comprises a substantially linear relationship during which the pressure in the cavity increases with respect to the delivery of insufflating gas to the cavity, and preferably, the second pressure/volume relationship comprises a substantially linear relationship, during which the value of the increase in pressure in the cavity per unit volume of insufflating gas delivered to the cavity remains at a substantially constant value, and preferably, at a substantially constant value greater than the substantially constant value of the increase in pressure in the cavity per unit volume of insufflating gas delivered to the cavity during the first pressure/volume relationship.

In another embodiment of the invention the first pressure/volume relationship transitions to the second pressure/volume relationship through an intermediate pressure/volume relationship, and preferably, the intermediate pressure/volume relationship comprises a non-linear relationship.

In another embodiment of the invention the transition pressure value is determined as a pressure value lying in a range between a first pressure value and a second pressure value, the first pressure value being determined as the pressure at which the first pressure/volume relationship transitions to the intermediate pressure/volume relationship, and the second pressure value being determined as the pressure at which the intermediate pressure/volume relationship transitions to the second pressure/volume relationship.

In one embodiment of the invention the transition pressure value is determined as the average value of the first and second pressure values.

In another embodiment of the invention the transition pressure value is determined as a point of inflection on a line of a graph representative of the pressure/volume relationship during insufflating of the cavity as the first pressure/volume relationship transitions to the second pressure/volume relationship.

In another embodiment of the invention in the graph representative of the pressure/volume relationship, volume is plotted on the abscissa, and pressure is plotted on the ordinate.

In one embodiment of the invention the point of inflection is determined by interpolating the point of intersection of a portion of the line representative of the first pressure/volume relationship and a portion of the line representative of the second pressure/volume relationship. In an alternative embodiment of the invention the portion of the line representative of the first pressure/volume relationship is extrapolated beyond the first pressure value, and the portion of the line representative of the second pressure/volume relationship is extrapolated beyond the second pressure value, and the point of inflection is determined as the point of intersection of the extrapolated portion of the line representative of the first pressure/volume relationship and the extrapolated portion of the line representative of the second pressure/volume relationship.

In one embodiment of the invention the value of the pressure in the cavity and the corresponding value of either the rate at which insufflating gas is being delivered to the cavity or the cumulative volume of insufflating gas delivered to the cavity are determined either continuously or at predefined time intervals.

In another embodiment of the invention each pair of the determined values of either the pressure in the cavity and the corresponding rate at which the insufflating gas is being delivered to the cavity, or the pressure in the cavity and the corresponding cumulative volume of insufflating gas delivered to the cavity are time-stamped, cross-referenced and stored.

In one embodiment of the invention the value of the transition pressure is determined from the pairs of the values of the pressure in the cavity and the corresponding rate at which insufflating gas is being delivered to the cavity.

In another embodiment of the invention the value of the transition pressure is determined from the stored, cross-referenced and time-stamped pairs of values of the pressure in the cavity and the corresponding cumulative volume of insufflating gas delivered to the cavity.

Preferably, the value of the increase in the pressure of the cavity per unit volume of insufflating gas delivered to the cavity is computed from each pair of the determined values of the pressure in the cavity and the corresponding rate of delivery of insufflating gas to the cavity, or from each pair of the determined values of the pressure in the cavity and the corresponding cumulative volume of insufflating gas delivered to the cavity. Preferably, a smoothing algorithm is applied to each computation of the increase in pressure in the cavity per unit volume of insufflating gas delivered to the cavity, and preferably, the smoothing algorithm comprises a moving average algorithm. Advantageously, each computed value of the increase in pressure in the cavity per unit increase in the volume of insufflating gas delivered to the cavity and the corresponding value of the pressure in the cavity are time-stamped, cross-referenced and stored.

In one embodiment of the invention the value of the transition pressure is determined from the computed values of the increase in pressure in the cavity per unit volume of insufflating gas delivered to the cavity and the corresponding values of the pressure in the cavity.

In another embodiment of the invention the first pressure value is determined from the computed values of the increase in the pressure in the cavity per unit volume of insufflating gas delivered to the cavity and the corresponding values of the pressure in the cavity.

In another embodiment of the invention the second pressure value is determined from the computed values of the increase in the pressure in the cavity per unit volume of insufflating gas delivered to the cavity and the corresponding values of the pressure in the cavity.

Preferably, the pressure/volume relationship is determined each time the value of the pressure in the cavity and the corresponding value of either the rate at which the insufflating gas is being delivered to the cavity or the cumulative volume of insufflating gas delivered to the cavity are determined.

In another embodiment of the invention delivery of insufflating gas to the cavity is terminated in response to the pressure in the cavity reaching a maximum safe pressure value beyond which the cavity can no longer be safely insufflated.

In one embodiment of the invention the maximum safe pressure value is selectable or predefined.

In another embodiment of the invention the maximum safe pressure value lies in the range of 25 mmHg to 30 mmHg. Advantageously, the maximum safe pressure value is approximately 30 mmHg.

In another embodiment of the invention delivery of insufflating gas to the cavity is terminated in response to the value of the transition pressure being determined.

In another embodiment of the invention delivery of insufflating gas to the cavity is terminated in response to the second pressure value being determined.

Preferably, the insufflating gas is delivered to the cavity at a relatively slow rate, while the value of the optimum maximum pressure is being determined.

In one embodiment of the invention a signal indicative of the value of the optimum maximum pressure is produced. Preferably, the signal indicative of the value of the optimum maximum pressure comprises a human sensory perceptible signal. Advantageously, the signal indicative of the value of the optimum maximum pressure is adapted for applying to a visual display screen, for display thereon. Preferably, the signal indicative of the value of the optimum maximum pressure is adapted for storing in an electronic memory of an insufflator.

Additionally, the invention provides a method for operating an insufflator for insufflating a cavity in the body of a human or animal subject, the insufflator comprising a delivery means for delivering insufflating gas to the cavity, and a signal processor for controlling the operation of the delivery means, the method comprising storing the value of the optimum maximum pressure value determined by the method according to the invention in an electronic memory of the insufflator, and programming the signal processor to control the delivery means to limit the delivery of insufflating gas to the cavity or to produce a signal adapted for applying to a means for producing a human sensory perceptible signal warning of the pressure in the cavity reaching the optimum maximum pressure, in response to the pressure in the cavity reaching the optimum maximum pressure value.

In one embodiment of the invention the signal processor is programmed to terminate delivery of insufflating gas to the cavity in response to the pressure in the cavity reaching the optimum maximum pressure value.

In another embodiment of the invention the delivery of insufflating gas to the cavity is reinstated on the pressure in the cavity falling below the optimum maximum pressure value.

In a further embodiment of the invention the insufflator comprises a pressure sensor for monitoring the pressure in the cavity, the pressure sensor being configured to produce a signal indicative of the pressure in the cavity, and the signal processor is programmed to control the delivery means to maintain the pressure in the cavity substantially at a selectable desired working pressure in response to the value of the signal indictive of the pressure in the cavity read from the pressure sensor.

The advantages of the invention are many. By knowing the value of the optimum maximum pressure value for a cavity of a subject, beyond which delivering further insufflating gas into the cavity results in little or no increase in the working volume in the cavity without a significant increase in cavity pressure, avoids a surgeon setting or increasing the pressure in the cavity beyond the optimum maximum pressure value in order to gain an increase in the working volume in the cavity. Additionally, if a surgeon has inadequate visualisation in a cavity, due to the working volume in the cavity, at a pressure below the optimum maximum pressure for that cavity, the surgeon knows that by increasing the pressure in the cavity, the working volume in the cavity will increase, and in turn improve visualisation provided that the optimum maximum pressure value is not exceeded. Additionally, if a surgeon has adequate visualisation at a cavity pressure, which for example, may be just at or just below the optimum maximum pressure value, the pressure in the cavity may be needlessly high, and the surgeon may reduce the cavity pressure to a lower pressure with a minimal decrease in the working volume in the cavity, and thus minimal decrease in visualisation. Accordingly, knowing the optimum maximum pressure value allows the cavity of a subject to be insufflated to the lowest possible insufflating pressure in order to obtain adequate visualisation, thereby avoiding any risk of over pressurisation of the cavity of the subject, and in turn avoiding any clinical risks, such as post operative pain, reduction in venous return and other such complications.

The invention will be more clearly understood from the following description of some preferred embodiments thereof, which are given by way of example only, with reference to the accompanying drawings, in which:

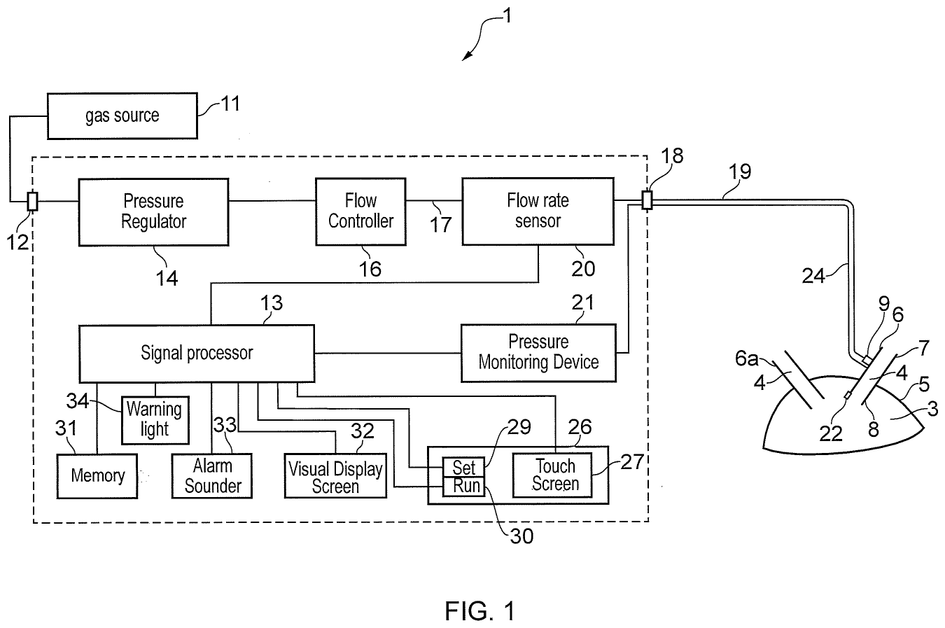

FIG. 1 is a block representation of an insufflator according to the invention, and

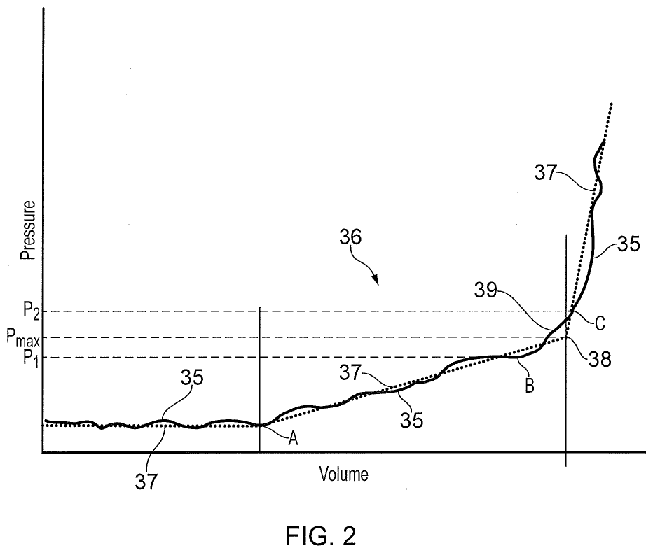

FIG. 2 is graphical representation of a pressure/volume relationship between pressure in the cavity in the body of a subject being insufflated by the insufflator of FIG. 1 and the cumulative volume of insufflating gas delivered to the cavity with volume plotted on the abscissa and pressure plotted on the ordinate.

Referring to the drawings there is illustrated an insufflator according to the invention indicated generally by the reference numeral 1 for insufflating a cavity, in this case the peritoneal cavity 3 in the body 5 of a human or animal subject during a minimally invasive surgical or investigative procedure being carried out, typically, laparoscopically. Although, it will be readily apparent to those skilled in the art that the insufflator 1 may be used for insufflating any lumen, vessel or cavity in the body of a human or animal subject, laparoscopically, endoscopically or otherwise. The insufflator 1 as will be described in detail below is operable in two modes, namely, in a normal insufflating mode and in a set-up mode.

In the normal insufflating mode the insufflator 1 delivers insufflating gas to the cavity 3 through a trocar 6 for insufflating the cavity 3 and for maintaining the cavity insufflated during the surgical or investigative procedure. In the set-up mode of the insufflator 1 an optimum maximum pressure value, above which the cavity 3 of the subject should ideally not be insufflated, is determined prior to commencement of operating of the insufflator 1 in the normal insufflating mode for insufflating the cavity 3 of that subject. The optimum maximum pressure value is a pressure at which any further delivery of insufflating gas into the cavity of the subject would result in a further increase of the pressure in the cavity with little or no further increase in the working volume in the cavity.

The trocar 6 comprises an instrument channel 4 extending therethrough, and extends from a proximal end 7 to a distal end 8, which is located in the cavity 3, and may or may not comprise an insufflating gas inlet port 9 to which insufflating gas is delivered to the trocar 6. A bore (not shown) extending along the wall of the trocar 6 from the inlet port 9 to the distal end 8 of the trocar 6 accommodates insufflating gas from the inlet port 9 into the cavity 3. Alternatively, the insufflating gas may be delivered through a Veress needle.

The insufflator 1 comprises a housing 10, which may include a source of insufflating gas, which if included in the housing 10 would normally be provided in a pressurised container containing pressurised insufflating gas, typically, carbon dioxide. However, in this embodiment of the invention the insufflator 1 is adapted to receive the pressurised insufflating gas from an external source 11, which typically, comprises compressed carbon dioxide, typically, from a source of compressed carbon dioxide available in a hospital operating theatre, or elsewhere where the insufflator 1 is being operated. An inlet port 12 is provided in the housing 10 for connecting the insufflator 1 to the external pressurised source 11 of the insufflating gas. A signal processor, in this case provided by a microprocessor 13 located in the housing 10 controls the operation of the insufflator 1 as will be described below. However, it will be appreciated that any other suitable form of signal processor may be provided, for example, a microcontroller or other such suitable signal processor.

A pressure regulator 14 located in the housing 10 is connected to the inlet port 12 for receiving the insufflating gas therefrom, and for stepping down the pressure of the insufflating gas to a pressure not exceeding 40 mmHg.

A delivery means comprising a flow controller 16 located in the housing 10, controls the flow rate of the insufflating gas to the cavity 3 of the subject, and in turn the pressure in the cavity 3. The flow controller 16 is connected to the pressure regulator 14 and receives the insufflating gas from the pressure regulator 14 at the stepped down pressure. The flow controller 16 is operated under the control of the microprocessor 13 for controlling the flow rate at which the insufflating gas is delivered to the cavity 3 of the subject as will be described below.

A connecting tube 17 connects the flow controller 16 to an outlet port 18 of the housing 10. A gas line 19 from the outlet port 18 supplies the insufflating gas to the cavity 3 of the subject through the trocar 6. The gas line 19 may be entered directly into the cavity 3 through the instrument channel 4 in the trocar 6, or if the trocar 6 is provided with the gas inlet port 9, the gas line 19 may be connected to the gas inlet port 9, through which the insufflating gas is delivered into the cavity 3.

A flow rate sensor 20 located in the housing 10 in the connecting tube 17 monitors the flow rate of the insufflating gas through the connecting tube 17 to the cavity 3, and produces a signal indicative of the rate at which the insufflating gas is being delivered to the cavity 3. The microprocessor 13 is programmed to read the value of the signal produced by the flow rate sensor 20 and to compute the cumulative volume of the insufflating gas delivered to the cavity 3 from the commencement of delivery of insufflating gas thereto.

A pressure monitoring device 21 located in the housing 10 monitors cavity pressure, namely, the pressure in the cavity 3 of the subject and produces a signal indicative of the pressure in the cavity 3. The pressure monitoring device 21 reads signals from a pressure sensor 22 which may be located in the housing 10 in the connecting tube 17 downstream of the flow sensor 20, or may be located in the cavity 3, for example, on a portion of the trocar 6 located within the cavity 3, which would give a direct reading of the cavity pressure. If the pressure sensor were located in the connecting tube 17 downstream of the flow sensor 20, during monitoring of the cavity pressure, in order to determine the cavity pressure, either the flow controller 16 would isolate the cavity 3 from the insufflating gas to give a true value of the pressure in the cavity 3, or alternatively, the pressure of the insufflating gas flowing through the connecting tube 17 would be read from the pressure sensor. The pressure monitoring device 21 would then apply a compensating factor to the pressure value read from the pressure sensor to obtain the true value of the cavity pressure. However, in order to determine the value of the compensating value to be applied to the signals indicative of the read pressure value of the insufflating gas flowing in the connecting tube 17, the pressure monitoring device would obtain the flow rate of the insufflating gas in the connecting tube 17 from either the flow sensor or the microprocessor 13, and would apply a suitable compensating value to the signal read from the pressure sensor taking account of the read pressure value, the flow rate of the insufflating gas in the connecting tube 17, as well as the frictional resistance to flow in the connecting tube 17 and the gas line 19 between the pressure sensor and the trocar 6.

Alternatively, the pressure sensor, if located in the housing 10 may be connected directly to the cavity 3 through a pressure monitoring conduit (not shown) which would extend from the pressure sensor through the trocar 6 into the cavity 3, or through a second trocar 6a, similar to the trocar 6 extending into the cavity 3, so that the pressure monitored by the pressure sensor would be the true pressure in the cavity 3.

In this embodiment of the invention the pressure sensor 22 is located on an outer surface of the trocar 6 adjacent the distal end 8 thereof, and produces a signal indicative of the cavity pressure. An electrically conductive wire 24 connects the pressure sensor 22 to the pressure monitoring device 21 to apply the signal from the pressure sensor 22 to the pressure monitoring device 21. The wire 24 extends along and is secured to the outer surface of the gas line 19 and enters the housing 10 with the gas line 19 at the outlet port 18, and then extends to and is connected to the pressure monitoring device 21. However, it is envisaged that in some embodiments of the invention the communication between the pressure sensor 22 and the pressure monitoring device 21 may be provided wirelessly.

The microprocessor 13 is programmed to control the flow controller 16 to control the rate of flow of insufflating gas to the cavity 3 to maintain the pressure in the cavity 3 at a selected working pressure, typically in the range of 10 mmHg to 15 mmHg, when the microprocessor 13 is controlling the insufflator 1 to operate in the normal insufflating mode.

An interface 26, which may comprise a touch screen, a keypad or any other suitable interface, and which in this case comprises a touch screen 27, is located on the housing 10 to enable entry into the microprocessor 13 of the value of the selected working pressure, at which the cavity pressure is to be maintained during insufflating of the cavity 3 when the insufflator 1 is operating in the normal insufflating mode, and other relevant data, for example, the maximum safe pressure to which the cavity 3 may be insufflated in the normal insufflating mode, and the maximum set-up safe pressure when the insufflator 1 is operating in the set-up mode. The maximum safe pressure in the normal insufflating mode and the maximum set-up safe pressure may be the same or different, and depending on the cavity being insufflated may range between 25 mmHg and 30 mmHg or between 10 mmHg and 15 mmHg. In some embodiments of the invention the maximum safe pressure may range between 20 mmHg and 25 mmHg. Although, in some embodiments of the invention the maximum safe pressure in the normal insufflating mode and the maximum set-up safe pressure may be pre-set in the microprocessor 15.

The interface 26 also comprises a pair of button switches, namely, a set-up mode select button switch 29 and a normal insufflating mode select button switch 30 for selecting the operational mode of the insufflator 1, namely, the set-up mode or the normal insufflating mode, respectively. The microprocessor 13 reads signals from the touch screen 27 and from the set-up mode and normal insufflating mode select button switches 29 and 30 of the interface 26.

A memory 31 of the microprocessor 13 which may be any suitable electronic memory such as a random access memory is provided for storing data for access by the microprocessor 13 as will be described below. The microprocessor 13 controls a visual display screen 32 which provides visual data to a surgeon and clinician personnel in an operating theatre. An alerting device for producing an alerting signal is also operated under the control of the microprocessor 13 in the event of the cavity pressure exceeding either of the maximum safe pressures, or as will be described below, the optimum maximum pressure. The alerting device, in this case comprises both an alarm sounder 33 and a warning light 34 mounted on the housing 10 for producing an audible alerting signal and a visual alerting signal, respectively.

Turning now to the operation of the insufflator 1, the insufflator 1 is connected to the external pressurised source 11 of insufflating gas through the inlet port 12. The gas line 19 is connected to the gas inlet port 9 of the trocar 6 which has already been inserted into the cavity 3 of the subject for supplying insufflating gas to the cavity through the trocar 6. Alternatively, the gas line 19 may be entered directly into the cavity 3 through the trocar 6. The wire 24 from the pressure sensor 22 is connected to the pressure monitoring device 21 through the outlet port 18 of the housing 10.

Initially, the insufflator 1 is operated in the set-up mode to determine the optimum maximum pressure, above which the cavity 3 of the subject should ideally not be inflated. The set-up select button switch 29 is operated to set the microprocessor 13 to operate in the set-up mode, and in turn to operate the insufflator 1 in the set-up mode. In the set-up mode, the microprocessor 13 controls the flow controller 16 to deliver insufflating gas to the cavity 3 of the subject at a constant rate, typically in the range of 0.5 litres per minute to 5 litres per minute, and reads the values of the signal from the flow sensor 20 and the values of the signal from the pressure monitoring device 21 at predefined time intervals, typically of 10 milliseconds as the insufflating gas is being slowly delivered to the cavity 3.

As the values of the signals are read from the flow sensor 20 and the pressure monitoring device 21 at the end of each predefined time interval, the microprocessor 13 is programmed to determine the cumulative volume of insufflating gas delivered to the cavity 3 from the commencement of delivery of the insufflating gas to the cavity 3 and the corresponding pressure in the cavity 3. The determined cumulative volume of insufflating gas delivered to the cavity 3 and the corresponding pressure in the cavity 3 are then time-stamped, cross-referenced and stored in the memory 31 of the microprocessor 13. The microprocessor 13 is programmed to compute a pressure/volume relationship between the pressure in the cavity 3 and the cumulative volume of insufflating gas delivered to the cavity 3 at the end of each predefined time interval, as each new value of the cumulative volume of the insufflating gas delivered to the cavity 3 and the corresponding value of pressure in the cavity 3 is determined, and each computed pressure/volume relationship is time-stamped and stored in the memory 31. Additionally, at the end of each predefined time interval, the microprocessor 13 is programmed to compute a value of the increase in pressure in the cavity 3 per unit volume of insufflating gas delivered to the cavity 3, and each computed value of the increase in the pressure in the cavity 3 per unit volume of insufflating gas delivered to the cavity 3 and the corresponding value of the pressure in the cavity are time-stamped, cross-referenced and stored in memory 31. The microprocessor 13 is programmed when computing the value of the increase in the pressure in the cavity per unit volume of insufflating gas delivered to the cavity to apply a smoothing algorithm to the computation to smooth the computed values. The microprocessor 13 is also programmed when computing each value of the pressure/volume relationship to apply a smoothing algorithm to the computation. In this embodiment of the invention the smoothing algorithms comprise respective moving average algorithms, although any other smoothing algorithms may be used.

Before describing the steps carried out by the microprocessor 13 in the set-up mode further to determine the optimum maximum pressure value, reference is now made to FIG. 2 in which a line 35 of a graph 36 represents a graphical relationship of the actual pressure/volume relationship between the cumulative volume of insufflating gas delivered to the cavity 3 and the pressure in the cavity 3. The pressure in the cavity 3 is plotted on the ordinate, and the cumulative volume of the insufflating gas delivered to the cavity 3 is plotted on the abscissa of the graph 36. A line 37 representing a smoothed version of the pressure/volume relationship of the line 35, after the moving average algorithm has been applied to the pressure/volume relationship of the line 35 is also illustrated in FIG. 2.

As can be seen from the lines 35 and 37, initially, as insufflating gas is being delivered to the cavity 3, the pressure of the cavity 3 remains substantially constant as the cumulative volume of the insufflating gas which is delivered to the cavity 3 increases, until the cavity 3 has been filled with the insufflating gas. At this point, namely, at the point A on the line 35, as further insufflating gas is delivered to the cavity 3, the cavity pressure commences to rise, thereby establishing a first pressure/volume relationship between the cavity pressure and the cumulative volume of insufflating gas delivered to the cavity 3. This first pressure/volume relationship continues from the point A to the point B, during which the first pressure/volume relationship is a substantially linear relationship with the pressure in the cavity 3 increasing as the cumulative volume of insufflating gas delivered to the cavity 3 increases, and with the value of the increase in pressure in the cavity 3 per unit volume of insufflating gas delivered to the cavity 3 being of a first substantially constant value, which is greater than zero. The slope of the line 35 represents the increase in pressure in the cavity 3 per unit volume of insufflating gas delivered to the cavity 3.

At point B on the line 35, the substantially linear first pressure/volume relationship transitions to an intermediate pressure/volume relationship, which is non-linear, and during which the value increase in the pressure in the cavity 3 per unit volume of insufflating gas delivered to the cavity 3 progressively increases. The non-linear intermediate pressure/volume relationship continues to point C on the line 35. At point C of the line 35, the non-linear intermediate pressure/volume relationship transitions to a second pressure/volume relationship comprising a substantially linear relationship with the pressure in the cavity 3 increasing as the cumulative volume of insufflating gas in the cavity 3 increases, and during which the value of the increase in the pressure in the cavity 3 per unit volume of insufflating gas delivered to the cavity 3 is of a second substantially constant value, which is significantly greater than the first substantially constant value of the increase in pressure in the cavity 3 per unit volume of insufflating gas delivered to the cavity 3 of the first pressure/volume relationship.

Thus, from point B on the line 35 at the end of the first pressure/volume relationship, the pressure in the cavity 3 commences to rise from a first pressure value P₁ at a non-linear ever-increasing rate per unit volume of gas delivered to the cavity 3 of the subject to a second pressure value P₂ at the point C on the line 35 at the commencement of the second pressure/volume relationship. Accordingly, the gain in working volume in the cavity 3 per unit volume of gas delivered to the cavity begins to decrease from the point B, while the increase in pressure per unit volume of insufflating gas delivered to the cavity 3 begins to increase. Thus, at some point between the first pressure value P₁ at point B of the line 35 and the second pressure value P₂ at point C of the line 35, and onward from the point C, the gain in working volume in the cavity 3 per unit volume of gas delivered to the cavity 3 is marginal while the pressure in the cavity 3 commences to rapidly increase.

The microprocessor 13 is programmed to determine the optimum maximum pressure at which the gain in working volume in the cavity 3 per unit volume of insufflating gas delivered to the cavity 3 with respect to the pressure in the cavity is optimised. In this embodiment of the invention the microprocessor 13 is programmed to determine the optimum maximum pressure as the pressure at the point of intersection 38 of the portion of the smoothed line 37 representative of the first pressure/volume relationship and the portion of the smoothed line 37 representative of the second pressure/volume relationship. The microprocessor 13 is programmed to determine the point of intersection 38 of the portions of the smoothed line 37 representative of the first pressure/volume relationship and the second pressure/volume relationship by either interpolating between the two portions representative of the first and second pressure/volume relationships, or by extrapolating the portion of the smoothed line 37 representative of the first pressure/volume relationship forward beyond the value of the pressure P₁, and by extrapolating the portion of the smoothed line 37 representative of the second pressure/volume relationship backwards from the value of the pressure P₂.

The point of intersection 38 on the line 37 approximates to the point of inflection of the line 35, at which the first pressure/volume relationship transitions to the second pressure/volume relationship, and the value of the pressure in the cavity 3 at the point of intersection 38 on the line 37 is the value of the transition pressure at which the first pressure/volume relationship transitions to the second pressure/volume relationship. The microprocessor 13 is programmed to determine the value of the pressure in the cavity 3 at the point of intersection 38 as the transition pressure value, and to store the value of the transition pressure in the memory 31 as the value of the optimum maximum pressure Pₘₐₓ for the cavity 3, above which the insufflator 1 should ideally not insufflate the cavity 3, since there is little or no gain in the working volume in the cavity 3 once the pressure in the cavity 3 reaches the value of the optimum maximum pressure Pₘₐₓ. The microprocessor 13 is programmed to output a signal indicative of the value of the optimum maximum pressure Pₘₐₓ to the visual display screen, and the value of the optimum maximum pressure Pₘₐₓ is displayed on the visual display screen 32 under the control of the microprocessor 13. Additionally, the microprocessor 13 may be programmed, when running in the normal insufflating mode, to control the flow controller 16 to prevent the pressure in the cavity 3 exceeding the optimum maximum pressure value, as will be described below.

If at any stage, while the insufflator 1 is operating in the set-up mode, the pressure in the cavity 3 exceeds the maximum set-up safe pressure value, the microprocessor 13 is programmed to operate the flow controller 16 to terminate delivery of insufflating gas to the cavity 3.

On the value of the optimum maximum pressure being determined, insufflating of the cavity 3 may be terminated, or insufflating of the cavity 3 may be continued, if the insufflator 1 is to be immediately switched to operate in the normal insufflating mode, as will be described below.

In an alternative embodiment of the invention the microprocessor 13 is programmed to determine the value of the optimum maximum pressure Pₘₐₓ, beyond which the cavity 3 should ideally not be insufflated, by determining the first pressure value P₁ at which the first pressure/volume relationship transitions to the intermediate pressure/volume relationship, and also by determining the second pressure value P₂ at which the intermediate pressure/volume relationship transitions to the second pressure/volume relationship, and then determining the average value of the first and second pressure values P₁ and P₂. In this embodiment of the invention the microprocessor 13 is programmed at the end of each predefined time interval to compute the value of the increase in the pressure in the cavity 3 per unit volume of insufflating gas delivered to the cavity 3, which is equivalent to the slope of the line 35 of the graph 36. However, in order to enable the first and second pressure values P₁ and P₂ to be determined more accurately, at the end of each predefined time interval, when computing the increase in the pressure in the cavity 3 per unit volume of insufflating gas delivered to the cavity 3, a smoothing algorithm, which in this case is also a moving average algorithm, is applied to the computation. Although, any suitable smoothing algorithm may be used. As each value of the increase in pressure in the cavity 3 per unit volume of insufflating gas delivered to the cavity is being computed, the corresponding value of the pressure in the cavity 3 is read and each corresponding pair of the values of the computed increase in pressure in the cavity per unit volume of insufflating gas delivered to the cavity and the pressure in the cavity are time-stamped, cross-referenced and stored in the memory 31.

As successive values of the increase in pressure in the cavity 3 per unit volume of insufflating gas delivered to the cavity 3 are computed, the signal processor is programmed to compare each computed value of the increase in the pressure in the cavity 3 per unit volume of insufflating gas delivered to the cavity 3 with the previously computed value or a number of the previously computed values of the increase in pressure in the cavity per unit volume of insufflating gas delivered to the cavity, to determine the first pressure value P₁. The microprocessor 13 is programmed to determine the first pressure value P₁ as the pressure in the cavity 3 when the computed values of the increase in pressure in the cavity 3 per unit volume of insufflating gas delivered to the cavity 3 ceases to be substantially constant. In other words, the first pressure value P1 is determined as the value of the pressure in the cavity 3 just prior to the value of the increase in pressure in the cavity per unit volume of insufflating gas delivered to the cavity beginning to increase.

Once the first pressure value P₁ has been determined, the microprocessor 13 is programmed to continue computing the values of the increase in the pressure in the cavity per unit volume of insufflating gas delivered to the cavity at the ends of the respective predefined time periods. On each value of the increase in pressure in the cavity 3 per unit volume of insufflating gas delivered to the cavity 3 being computed, the microprocessor 13 is programmed to compare the computed value with the previous or a number of the previously computed values of the increase in the pressure in the cavity per unit volume of insufflating gas delivered to the cavity. The microprocessor 13 is programmed to determine the second pressure value P₂ as being the pressure in the cavity 3 at which the first of the computed values of the increase in the pressure in the cavity 3 per unit volume of insufflating gas delivered to the cavity 3 becomes substantially constant again.

When the first and second pressure values P₁ and P₂ have been determined, the microprocessor 13 is programmed to determine the average value of the first and second pressure values P₁ and P₂, and the average value of the first and second pressure values P₁ and P₂ is determined as being the value of the optimum maximum pressure Pₘₐₓ beyond which the cavity 3 should ideally not be insufflated.

Once the value of the optimum maximum pressure Pₘₐₓ has been determined, the insufflator 1 is switched from the set-up mode to the normal insufflating mode by operating the normal insufflating select button switch 30 to operate the insufflator 1, and in turn, the microprocessor 13, in the normal insufflating mode. If the desired value of the working pressure at which the cavity 3 is to be insufflated and the maximum safe pressure to which the cavity 3 may be safely insufflated during operation of the insufflator 1 in the normal insufflating mode have not yet been entered, both the desired value of the working pressure and the value of the maximum safe pressure are entered in the microprocessor 13 through the touch screen 27. The desired working pressure will, in general, be selected as a pressure value below the value of the optimum maximum pressure.

In the normal insufflating mode, the microprocessor 13 controls the operation of the flow controller 16 to supply insufflating gas to the cavity so that the pressure in the cavity 3 is maintained at the selected desired working pressure. The microprocessor 13 reads the value of the signal produced by the pressure monitoring device 21 and controls the flow controller 16 to deliver insufflating gas to the cavity at the appropriate flow rate in order to maintain the pressure in the cavity at the selected desired working pressure.

If the surgeon inputs a signal through the touchscreen 27 requesting an increase in the working pressure in the cavity, the microprocessor 13 controls the flow controller 16 to increase the flow rate of insufflating gas to the cavity 3 to increase the pressure in the cavity 3. The microprocessor 13 controls the operation of the flow controller 16 to increase the flow rate of the insufflating gas to the cavity 3 in incremental steps and reads the value of the signal produced by the pressure monitoring device 21 to check that the pressure in the cavity 3 is raising as requested. The microprocessor 13 controls the flow controller 16 in response to the value of the signal read from the pressure monitoring device 21 until the working pressure has been raised to the working pressure selected by the surgeon or until the surgeon indicates that no further increase in the pressure in the cavity 3 is required.

During raising of the pressure in the cavity 3, at the predefined time intervals, the microprocessor 13 compares the value of the pressure in the cavity 3 read from the pressure monitoring device 21 with the stored value of the optimum maximum pressure. In the event of the pressure in the cavity 3 reaching the optimum maximum pressure, the microprocessor 13 outputs an alert signal to the sounder 33 and the warning light 34 to alert the surgeon to the fact that the pressure in the cavity 3 has reached the optimum maximum pressure. The microprocessor 13 also outputs the alert signal to the visual display screen 32 for displaying the pressure in the cavity 3 on the visual display screen 32, and also controls the visual display screen 32 to present a message indicating that the pressure in the cavity 3 has reached the optimum maximum pressure. The microprocessor 13 operates the flow controller 16 to terminate the supply of insufflating gas to the cavity 3 until the pressure in the cavity 3 has fallen back to the optimum maximum pressure.

Additionally, the microprocessor 13 may be programmed to allow a surgeon override the signal to the flow controller 16 terminating supply of insufflating gas to the cavity 3, and to allow the microprocessor 13 to operate the flow controller 16 to deliver insufflating gas to the cavity for further rising the pressure therein above the optimum maximum pressure value.

However, should the pressure in the cavity be raised to a level where the pressure in the cavity reaches the maximum safe pressure, or the maximum set-up pressure when the insufflator is operating in the set-up mode, the microprocessor 13 outputs a further alert signal to the sounder 33 and the warning light 34 to warn the surgeon that the pressure in the cavity 3 has reached the maximum safe pressure, or the maximum set-up safe pressure, as the case may be, and also outputs a signal to the visual display screen 32 for displaying the current pressure in the cavity 3 along with a warning message warning the surgeon that the pressure in the cavity 3 has reached either of the maximum safe working pressures. The microprocessor 13 may be programmed to operate the flow controller 16 to terminate delivery of insufflating gas to the cavity 3 until the cavity pressure has fallen below the relevant maximum safe pressure.

In another embodiment of the invention the microprocessor may be programmed to allow insufflating of the cavity 3 beyond the maximum safe pressure, but only by direct intervention by the surgeon inputting an appropriate signal through the touchscreen 27.

On completion of the procedure, insufflating of the cavity 3 is terminated, and the insufflating gas is evacuated from the cavity. The gas line 19 is disconnected from the trocar 6 or withdrawn through the trocar 6. The wire 24 is also disconnected from the trocar 6 and from the pressure sensor 22 mounted on the trocar 6.

While the optimum maximum pressure value has been described as being the cavity pressure at the point of inflection between the portions of the smoothed line 37 of the graph 36 representing the first and second pressure/volume relationships, it is envisaged that the optimum maximum pressure value may be determined as being a pressure anywhere on the line 35 of the graph 36 between the first pressure value P₁ at point B and the second pressure value P₂ at point C on the line 35 of the graph, and may be a pressure closer to the first pressure value P₁ at point B on the line 35 than to the second pressure value P₂ at point C.

Needless to say, other methods for determining the point of inflection of the line 35 or the points at which the transitions from the first pressure/volume relationship to the intermediate pressure/volume relationship, or from the intermediate pressure/volume relationship to the second pressure/volume relationship occur, may be used besides those described.

While the pressure/volume relationship between the pressure in the cavity and the cumulative volume of insufflating gas delivered to the cavity has been described as comprising first and second pressure/volume relationships, in which the slopes of the lines representing the first and second pressure/volume relationships are substantially constant, and in which the value of the slope of the second pressure/volume relationship is greater than the value of the slope of the first pressure/volume relationship, and while the pressure/volume relationship between the first and second pressure/volume relationships is a non-linear intermediate relationship, it will be appreciated by those skilled in the art that the pressure/volume relationship between the pressure in the cavity and the corresponding cumulative volume of insufflating gas delivered to the cavity, may be other than such a pressure/volume relationship. For example, in some embodiments of the invention it is envisaged that the pressure/volume relationship may be in the form of an equation, such as a quadratic equation or other equation, which, for example, may be a power law equation. In which case, the line of the equation would be determined by, for example, curve fitting, and the slope of the line of the equation would be monitored in order to determine the point of inflection of the line at which the pressure/volume relationship transitions from the first pressure/volume relationship to the second pressure/volume relationship.

While the method and insufflator have been described for use in insufflating the peritoneal cavity of a subject, it will be readily apparent to those skilled in the art that the method and insufflator may be used for insufflating any cavity, lumen or vessel in the human or animal body, and for determining the optimum maximum pressure value for any cavity, lumen or vessel in the body of a human or animal subject.

It will also be appreciated that other suitable insufflating gases besides carbon dioxide may be used.

While the interface means has been described as comprising a touch screen, and set-up and normal insufflating select button switches, any other suitable interface means may be provided, for example, a keypad, and in some embodiments of the invention the insufflator may be operated remotely, for example, wirelessly from a smart mobile device, for example, a smart mobile phone.

While the flow sensor may be configured to produce a signal indicative of the cumulative volume of the insufflating gas delivered to the cavity, or may be configured to produce a signal indicative of the flow rate of the insufflating gas being delivered to the cavity, in either case, it is envisaged that the microprocessor would be appropriately programmed to read the signals from the flow sensor at predefined time intervals, typically, of 10 milliseconds each, and would be programmed to determine the appropriate pressure/volume relationship, and the values of the increase in the pressure in the cavity per unit volume of insufflating gas delivered to the cavity.

While the pressure regulator has been described as stepping down the pressure of the insufflating gas to 40 mmHg, it will be appreciated by those skilled in the art that the pressure regulator may step the pressure of the insufflating gas down to any suitable pressure, and in some embodiments of the invention the pressure to which the insufflating gas is stepped down by the pressure regulator may be less than or greater than 40 mmHg. Indeed, in some embodiments of the invention the pressure regulator may be configured to step down the pressure of the insufflating gas to a pressure significantly higher than the pressure of 40 mmHg, and in some cases, may only reduce the pressure of the insufflating gas to a pressure in the order of 3.5 bar. In which case, the flow controller would be configured to reduce the pressure from such a pressure to a suitable pressure, such that the insufflating gas would be supplied to the cavity at the appropriate pressure, such that during the procedure, the pressure in the cavity would be maintained at the selected working pressure, or at any other pressure chosen or selected by the surgeon or clinician. During initial insufflating of the cavity in order to determine the set pressure value, the flow controller would be operated to supply the insufflating gas to the cavity at the appropriate constant rate.

1-84. (canceled)

85. An insufflator adapted to be selectively operated in a normal insufflating mode and in a set-up mode, the insufflator being configured in the set-up mode for determining the value of an optimum maximum pressure for insufflating a cavity in the body of a human or animal subject, the insufflator comprising:
a delivery means for delivering insufflating gas to the cavity,
a pressure sensor for producing a signal indicative of the pressure in the cavity,
a flow sensor for monitoring flow of insufflating gas being delivered to the cavity and for producing a signal indicative of the cumulative volume of insufflating gas delivered to the cavity from the commencement of delivery of the insufflating gas thereto or a signal indicative of the rate at which the insufflating gas is being delivered to the cavity, and
a signal processor adapted to read the signal produced by the pressure sensor, and to read the signal produced by the flow sensor during insufflating of the cavity, and in the set-up mode:
to determine a pressure/volume relationship between the pressure in the cavity and insufflating gas delivered to the cavity as the insufflating gas is being delivered to the cavity from values of the signals read from the pressure sensor and the flow sensor, and
to determine the value of the optimum maximum pressure as the value of a transition pressure at which the pressure/volume relationship transitions from a first pressure/volume relationship to a second pressure/volume relationship, the second pressure/volume relationship being different to the first pressure/volume relationship.

86. An insufflator as claimed in claim 85 in which the pressure/volume relationship determined by the signal processor comprises the value of the increase in pressure in the cavity per unit volume of insufflating gas delivered to the cavity, and preferably, the first pressure/volume relationship comprises either a substantially linear relationship or a non-linear relationship, and preferably, the first pressure/volume relationship comprises a substantially linear relationship during which the pressure in the cavity increases linearly with respect to the delivery of insufflating gas to the cavity.

87. An insufflator as claimed in claim 85 in which the second pressure/volume relationship comprises either a substantially linear relationship or a non-linear relationship, and preferably, the second pressure/volume relationship comprises a substantially linear relationship during which the pressure in the cavity increases linearly with respect to the delivery of insufflating gas to the cavity, and advantageously, the value of the increase of the pressure in the cavity per unit volume of insufflating gas delivered to the cavity in the second pressure/volume relationship, is greater than the value of the increase in the pressure in the cavity per unit volume of insufflating gas delivered to the cavity in the first pressure/volume relationship.

88. An insufflator as claimed in claim 85 in which the first pressure/volume relationship transitions to the second pressure/volume relationship through an intermediate pressure/volume relationship, and preferably, the intermediate pressure/volume relationship comprises a non-linear relationship, and advantageously, the signal processor is programmed to determine the transition pressure value as a pressure value lying in a range between a first pressure value and a second pressure value, and the signal processor is programmed to determine the first pressure value as the pressure at which the first pressure/volume relationship transitions to the intermediate pressure/volume relationship, and the signal processor is programmed to determine the second pressure value as the pressure at which the intermediate pressure/volume relationship transitions to the second pressure/volume relationship, and preferably, the signal processor is programmed to determine the transition pressure value as the average value of the first and the second pressure values.

89. An insufflator as claimed in claim 85 in which the signal processor is programmed to determine the transition pressure value as a point of inflection on a line representative of a graph of the pressure/volume relationship during insufflating of the cavity, as the first pressure/volume relationship transitions to the second pressure/volume relationship, and preferably, in the graph of the pressure/volume relationship, volume is plotted on the abscissa, and pressure is plotted on the ordinate of the graph, and advantageously, the signal processor is programmed to determine the point of inflection by interpolating the point of intersection of a portion of the line representative of the first pressure/volume relationship and a portion of the line representative of the second pressure/volume relationship, and preferably, the signal processor is programmed to determine the point of inflection on the line of the graph representative of the pressure/volume relationship during insufflating of the cavity by extrapolating the portion of the line representing the first pressure/volume relationship beyond the first pressure value, and extrapolating the line representing the second pressure/volume relationship beyond the second pressure value, and to determine the value of the transition pressure at the point of intersection of the extrapolated parts of the lines representing the first and second pressure/volume relationship.

90. An insufflator as claimed in claim 85 in which the signal processor is programmed to read the values of the signals produced by the pressure sensor and the flow sensor either continuously or at predefined time intervals, and preferably, the signal processor is programmed to time-stamp, cross-reference and store in memory each pair of the values of the signals read from the pressure sensor and the flow sensor, and advantageously, the signal processor is programmed to determine the value of the transition pressure from the stored, time-stamped and cross-referenced values of the pairs of values of the signals read from the pressure sensor and the flow sensor, and preferably, the signal processor is programmed to determine the value of the cumulative volume of insufflating gas delivered to the cavity and the corresponding value of the pressure in the cavity, each time the values of the signals are read from the flow sensor and the pressure sensor, and to time-stamp, cross-reference and store in memory each pair of the determined value of the cumulative volume of the insufflating gas delivered to the cavity and the corresponding value of the pressure in the cavity, and advantageously, the signal processor is programmed to determine the value of the transition pressure from the stored, cross-referenced and time-stamped pairs of values of the cumulative volume of insufflating gas delivered to the cavity and the corresponding pressure in the cavity, and preferably, the signal processor is programmed to compute the value of the increase in the pressure in the cavity per unit volume of insufflating gas delivered to the cavity each time the values of the signals are read by the signal processor from the pressure sensor and the flow sensor, and advantageously, the signal processor is programmed to apply a smoothing algorithm to the computation of each value of the increase in pressure in the cavity per unit volume of insufflating gas delivered to the cavity.

91. An insufflator as claimed in claim 90 in which the signal processor is programmed to time-stamp, cross-reference and store in memory each computed value of the increase in the pressure in the cavity per unit volume of insufflating gas delivered to the cavity and the corresponding values of the pressure in the cavity, and advantageously, the signal processor is programmed to determine the value of the transition pressure from the computed values of the increase in pressure in the cavity per unit volume of insufflating gas delivered to the cavity and the corresponding values of the pressure in the cavity, and preferably, the signal processor is programmed to determine the first pressure value from the computed values of the increase in the pressure in the cavity per unit volume of the insufflating gas delivered to the cavity and the corresponding values of the pressure in the cavity, and advantageously, the signal processor is programmed to determine the second pressure value from the computed values of the increase in the pressure in the cavity per unit volume of the insufflating gas delivered to the cavity and the corresponding values of the pressure in the cavity, and preferably, the signal processor is programmed to store the value of the optimum maximum pressure in memory, and advantageously, the signal processor is programmed to produce a signal indicative of the value of the optimum maximum pressure, and preferably, the signal produced by the signal processor indicative of the value of the optimum maximum pressure is adapted for conversion to a human sensory perceptible signal, and advantageously, the signal indicative of the value of the optimum maximum pressure produced by the signal processor is adapted for applying to a visual display screen for displaying the value of the optimum maximum pressure thereon.

92. An insufflator as claimed in claim 85 in which the signal processor is programmed to terminate delivery of insufflating gas to the cavity in the set-up mode in response to the pressure in the cavity reaching a pressure beyond which the cavity can no longer be safely insufflated, and preferably, the signal processor is programmed to terminate delivery of insufflating gas to the cavity in the set-up mode in response to the cavity pressure reaching a maximum set-up safe pressure, and advantageously, the maximum set-up safe pressure is either selectable or predefined, and preferably, the maximum set-up safe pressure lies in the range of 20 mmHg to 25 mmHg, and advantageously, the maximum set-up safe pressure lies in the range of 10 mmHg to 15 mmHg, and preferably, the maximum set-up safe pressure is approximately 15 mmHg, and advantageously, the signal processor is programmed to terminate delivery of insufflating gas to the cavity in the set-up mode in response to the value of the transition pressure being determined, and preferably, the signal processor is programmed to terminate delivery of insufflating gas to the cavity in the set-up mode in response to the second pressure value being determined.

93. An insufflator as claimed in claim 85 in which the signal processor is programmed to limit the supply of insufflating gas to the cavity in response to the pressure in the cavity reaching the optimum maximum pressure value when the insufflator is operating in the normal insufflating mode, and preferably, the signal processor is programmed to terminate the supply of insufflating gas to the cavity in response to the pressure in the cavity exceeding the optimum maximum pressure when the insufflator is operating in the normal insufflating mode, and advantageously, the signal processor is programmed to terminate the supply of insufflating gas to the cavity in response to the pressure in the cavity reaching the optimum maximum pressure value when the insufflator is operating in the normal insufflating mode, and preferably, the signal processor is programmed to reinstate the supply of insufflating gas to the cavity in response to the pressure in the cavity falling below the optimum maximum pressure value when the insufflator is operating in the normal insufflating mode.

94. A method for determining an optimum maximum pressure value for insufflating a cavity in the body of a human or animal subject, the method comprising:
delivering insufflating gas to the cavity,
determining a pressure/volume relationship between the pressure in the cavity and insufflating gas delivered to the cavity as the insufflating gas is being delivered to the cavity,
determining the optimum maximum pressure value as the value of a transition pressure at which the pressure/volume relationship transitions from a first pressure/volume relationship to a second pressure/volume relationship, the second pressure/volume relationship being different to the first pressure/volume relationship.

95. A method as claimed in claim 94 in which the determined pressure/volume relationship comprises the value of the increase in pressure in the cavity per unit volume of insufflating gas delivered to the cavity, and preferably, the first pressure/volume relationship comprises either a substantially linear relationship or a non-linear relationship, and preferably, the first pressure/volume relationship comprises a substantially linear relationship during which the pressure in the cavity increases with respect to the delivery of insufflating gas to the cavity.

96. A method as claimed in claim 94 in which the second pressure/volume relationship comprises either a substantially linear relationship or a non-linear relationship, and preferably, the second pressure/volume relationship comprises a substantially linear relationship during which the pressure in the cavity increases with respect to the delivery of insufflating gas to the cavity, and advantageously, the value of the increase in the pressure in the cavity per unit volume of insufflating gas delivered to the cavity in the second pressure/volume relationship is greater than the value of the increase in the pressure in the cavity per unit volume of insufflating gas delivered to the cavity in the first pressure/volume relationship, and preferably, the first pressure/volume relationship transitions to the second pressure/volume relationship through an intermediate pressure/volume relationship, and preferably, the intermediate pressure/volume relationship comprises a non-linear relationship.

97. A method as claimed in claim 96 in which the transition pressure value is determined as a pressure value lying in a range between a first pressure value and a second pressure value, the first pressure value being determined as the pressure at which the first pressure/volume relationship transitions to the intermediate pressure/volume relationship, and the second pressure value being determined as the pressure at which the intermediate pressure/volume relationship transitions to the second pressure/volume relationship, and preferably, the transition pressure value is determined as the average value of the first and second pressure values.

98. A method as claimed in claim 94 in which the transition pressure value is determined as a point of inflection on a line of a graph representative of the pressure/volume relationship during insufflating of the cavity as the first pressure/volume relationship transitions to the second pressure/volume relationship, and preferably, in the graph representative of the pressure/volume relationship, volume is plotted on the abscissa, and pressure is plotted on the ordinate, and advantageously, the point of inflection is determined by interpolating the point of intersection of a portion of the line representative of the first pressure/volume relationship and a portion of the line representative of the second pressure/volume relationship, and preferably, the portion of the line representative of the first pressure/volume relationship is extrapolated beyond the first pressure value, and the portion of the line representative of the second pressure/volume relationship is extrapolated beyond the second pressure value, and the point of inflection is determined as the point of intersection of the extrapolated portion of the line representative of the first pressure/volume relationship and the extrapolated portion of the line representative of the second pressure/volume relationship.

99. A method as claimed in claim 94 in which the value of the pressure in the cavity and the corresponding value of either the rate at which insufflating gas is being delivered to the cavity or the cumulative volume of insufflating gas delivered to the cavity are determined either continuously or at predefined time intervals, and preferably, each pair of the determined values of either the pressure in the cavity and the corresponding rate at which the insufflating gas is being delivered to the cavity, or the pressure in the cavity and the corresponding cumulative volume of insufflating gas delivered to the cavity are time-stamped, cross-referenced and stored, and advantageously, the value of the transition pressure is determined from the pairs of the values of the pressure in the cavity and the corresponding rate at which insufflating gas is being delivered to the cavity, and preferably, the value of the transition pressure is determined from the stored, cross-referenced and time-stamped pairs of values of the pressure in the cavity and the corresponding cumulative volume of insufflating gas delivered to the cavity, and advantageously, the value of the increase in the pressure of the cavity per unit volume of insufflating gas delivered to the cavity is computed from each pair of the determined values of the pressure in the cavity and the corresponding rate of delivery of insufflating gas to the cavity, or from each pair of the determined values of the pressure in the cavity and the corresponding cumulative volume of insufflating gas delivered to the cavity, and preferably, a smoothing algorithm is applied to each computation of the increase in pressure in the cavity per unit volume of insufflating gas delivered to the cavity.

100. A method as claimed in claim 99 in which each computed value of the increase in pressure in the cavity per unit increase in the volume of insufflating gas delivered to the cavity and the corresponding values of the pressure in the cavity are time-stamped, cross-referenced and stored, and preferably, the value of the transition pressure is determined from the computed values of the increase in pressure in the cavity per unit volume of insufflating gas delivered to the cavity and the corresponding values of the pressure in the cavity, and advantageously, the first pressure value is determined from the computed values of the increase in the pressure in the cavity per unit volume of insufflating gas delivered to the cavity and the corresponding values of the pressure in the cavity, and preferably, the second pressure value is determined from the computed values of the increase in the pressure in the cavity per unit volume of insufflating gas delivered to the cavity and the corresponding values of the pressure in the cavity, and advantageously, the pressure/volume relationship is determined each time the value of the pressure in the cavity and the corresponding value of either the rate at which the insufflating gas is being delivered to the cavity or the cumulative volume of insufflating gas delivered to the cavity are determined, and preferably, delivery of insufflating gas to the cavity is terminated in the set-up mode in response to the pressure in the cavity reaching a maximum set-up safe pressure value beyond which the cavity can no longer be safely insufflated, and advantageously, the maximum set-up safe pressure value is selectable or predefined, and preferably, the maximum set-up safe pressure value lies in the range of 20 mmHg to 25 mmHg, and advantageously, the maximum set-up safe pressure value lies in the range of 10 mmHg to 15 mmHg, and preferably, the maximum set-up safe pressure value is approximately 15 mmHg.

101. A method as claimed in claim 94 in which delivery of insufflating gas to the cavity is terminated in response to the value of the transition pressure being determined, and preferably, delivery of insufflating gas to the cavity is terminated in response to the second pressure value being determined, and preferably, the insufflating gas is delivered to the cavity at a relatively slow rate, while the value of the optimum maximum pressure is being determined.

102. A method as claimed in claim 94 in which a signal indicative of the value of the optimum maximum pressure is produced, and preferably, the signal indicative of the value of the optimum maximum pressure comprises a human sensory perceptible signal, and advantageously, the signal indicative of the value of the optimum maximum pressure is adapted for applying to a visual display screen, for display thereon, and preferably, the signal indicative of the value of the optimum maximum pressure is adapted for storing in an electronic memory of an insufflator.

103. A method for operating an insufflator for insufflating a cavity in the body of a human or animal subject, the insufflator comprising a delivery means for delivering insufflating gas to the cavity, and a signal processor for controlling the operation of the delivery means, the method comprising storing the value of the optimum maximum pressure value determined by the method as claimed in claim 94 in an electronic memory of the insufflator, and programming the signal processor to control the delivery means to limit the delivery of insufflating gas to the cavity or to produce a signal adapted for applying to a means for producing a human sensory perceptible signal warning of the pressure in the cavity reaching the optimum maximum pressure, in response to the pressure in the cavity reaching the optimum maximum pressure value.

104. A method as claimed in claim 103 in which the signal processor is programmed to terminate delivery of insufflating gas to the cavity in response to the pressure in the cavity reaching the optimum maximum pressure value, and preferably, the delivery of insufflating gas to the cavity is reinstated on the pressure in the cavity falling below the optimum maximum pressure value, and advantageously, the insufflator comprises a pressure sensor for monitoring the pressure in the cavity, the pressure sensor being configured to produce a signal indicative of the pressure in the cavity, and the signal processor is programmed to control the delivery means to maintain the pressure in the cavity substantially at a selectable desired working pressure in response to the value of the signal indictive of the pressure in the cavity read from the pressure sensor.
